(19) United States
(12) Patent Application Publication
(10) Pub. No.: US 2026/0294766 A1
(43) Pub. Date: Oct. 1, 2026

(54) COLOUR-STABLE LIPSTICK WITH SWEETENER

(71) Applicant: BEIERSDORF AG, Hamburg (DE)

(72) Inventors: Tanja HARTWIG, Bad Oldeslohe (DE); Sepideh RESHAD, Hamburg (DE)

(73) Assignee: BEIERSDORF AG, Hamburg (DE)

(21) Appl. No.: 19/477,022

(22) PCT Filed: Apr. 9, 2024
(86) PCT No.: PCT/EP2024/059570

§ 371 Date: Oct. 20, 2025

(30) Foreign Application Priority Data
Apr. 21, 2023 (DE) 10 2023 203 700.9

(51) Int. Cl. A61K 8/49; A61K 8/34; A61K 8/365; A61K 8/60; A61K 8/67; A61K 8/85; A61K 8/92; A61Q 1/06

(52) U.S. Cl. CPC A61K 8/49; A61K 8/345; A61K 8/365; A61K 8/602; A61K 8/678; A61K 8/85; A61K 8/922; A61Q 1/06; A61K 2800/43

(57) ABSTRACT

The invention relates to a cosmetic lipstick containing a combination of a) sodium saccharin (INCI: Sodium Saccharin) and b) citric acid (INCI: Citric Acid).

The present invention relates to a cosmetic lipstick comprising a combination of sodium saccharin (INCI: Sodium Saccharin) and citric acid (INCI: Citric Acid).

The desire to look beautiful and attractive has been rooted in humans for thousands of years. Although ideals of beauty have changed over time, the pursuit of a flawless appearance has always been aimed for by humans, since a pleasant appearance increases their self-esteem and attractiveness to their fellow human beings.

The term “decorative cosmetics” comes from the Latin “decoratio”—the emphasizing of beauty. In most cases, colorants are used to emphasize individual parts of the body, especially on the face, and to lessen color inconsistencies.

In addition to face powders and rouge as powdered cosmetics and stick-shaped preparations, creamy preparations such as day creams and cream make-up based on emulsions are used for this purpose.

Lipstick is one of the most used decorative cosmetics. More than 50% of women in Germany use it. In addition to accentuating the color of the lips, it is usually also used for lip care.

Lipsticks generally consist of a wax matrix in which high concentrations of liquid and semi-solid oils and also pigments and fillers are incorporated. They are in solid stick form.

Lipsticks are subject to particularly high requirements with regard to their safety in terms of health, as it cannot be ruled out that parts of the preparation will come into contact with the mouth and/or be swallowed when they are applied and during wear. Since individual ingredients in lipsticks have an unpleasant intrinsic taste, it is additionally important to mask this intrinsic taste in order to make the lipsticks more attractive for the consumers. Sweeteners are often used for this purpose. Potentially usable sweeteners would in principle also include sodium saccharin (INCI: Sodium Saccharin) and/or neohesperidin dihydrochalcone (INCI: Neohesperidin Dihydrochalcone).

However, a disadvantage of the prior art is the fact that lipsticks with sodium saccharin tend to cause yellow discoloration (“yellow tinge”). Preparations with a combination of sodium saccharin and neohesperidin dihydrochalcone tend to cause red discoloration or even brown discoloration. This effect occurs particularly in the case of relatively high temperatures and relatively long storage periods. If there are (unplanned) time delays during production in the heating phase or if a lipstick is stored at a relatively high temperature (for example in summer), this can result in undesirable discoloration of the product. Under certain circumstances, the preparation has to be discarded entirely and disposed of at great expense during the production process.

It was therefore the object of the present invention to develop sweetener-containing lipstick formulations (in particular those with sodium saccharin and/or neohesperidin dihydrochalcone) that are color-stable even in the case of relatively high temperatures and relatively long storage periods.

Lipstick preparations often comprise one or more color pigments, since they are generally used to color the skin of the lips.

A disadvantage of the prior art is then the fact that the combination of sweetener (in particular in the case of sodium saccharin and neohesperidin dihydrochalcone) and pigments results in the preparations becoming inhomogeneous. It is noticeable even during production that in sweetener-containing formulations, the pigments are no longer homogeneously distributed in the hot, liquid stick compound, but instead sink to the bottom. Sticks with a uniform composition can thus no longer be cast from this liquid stick compound.

It was therefore the object of the present invention to develop a color pigment- and sweetener-containing lipstick formulation in which the color pigments can be homogeneously incorporated in the preparation.

The objects are surprisingly achieved by a cosmetic lipstick comprising a combination of
a) sodium saccharin (INCI: Sodium Saccharin) and
b) citric acid (INCI: Citric Acid).

It is preferred according to the invention here if the preparation comprises water and/or glycerin.

The objects are surprisingly also achieved by a process for producing a cosmetic lipstick, which is characterized in that
i) firstly sodium saccharin (INCI: Sodium Saccharin) is dissolved in water and
ii) in a separate vessel citric acid (INCI: Citric Acid) is dissolved in water,
iii) in a second step both aqueous solutions are successively combined with the fat phase and
iv) the combined solutions are mixed with the remaining ingredients of the preparation and homogenized.

Within the scope of the present disclosure, designations as “according to the invention”, “advantageous according to the invention” etc. always relate to the preparation according to the invention and the process according to the invention, unless stated otherwise.

Embodiments of the present invention that are advantageous according to the invention are thus characterized in that the lipstick preparation comprises sodium saccharin in a concentration of 0.2% to 10% by weight, based on the total weight of the preparation. A concentration of less than 1.5% by weight, based on the total weight of the preparation, is preferred according to the invention here if sodium saccharin is the sole sweetener in the formulation.

It is also advantageous according to the invention if the lipstick preparation comprises citric acid in a concentration of at least 0.05% by weight, based on the total weight of the preparation.

A concentration of at least 0.25% by weight, based on the total weight of the preparation, is preferred according to the invention here if sodium saccharin is the sole sweetener in the formulation.

If the preparation also comprises neohesperidin dihydrochalcone and color pigments in addition to sodium saccharin, then a concentration of citric acid of at least 0.1% by weight, based on the total weight of the preparation, is preferred according to the invention.

In addition to preparations comprising sodium saccharin as the sole sweetener, also advantageous according to the invention is a second embodiment which is characterized in that the lipstick preparation additionally comprises neohesperidin dihydrochalcone (INCI: Neohesperidin Dihydrochalcone).

In such a case, it is advantageous according to the invention if the lipstick preparation additionally comprises neohesperidin dihydrochalcone (INCI: Neohesperidin Dihydrochalcone) in a concentration of 0.1% to 5% by weight, based on the total weight of the preparation.

In such a case, the process according to the invention is characterized in that in step i) the neohesperidin dihydrochalcone is dissolved together with the sodium saccharin in the water/glycerin mixture.

It is preferred according to the invention in all cases if the water/glycerin mixture does not comprise more than 25% by weight of glycerin, based on the water/glycerin mixture.

Embodiments of the present invention that are advantageous according to the invention are also characterized in that the lipstick preparation comprises one or more colorants selected from the group of CI15850 (also Red 6, E180 or Lithol Rubine), CI42090 (also Blue 1, E133 or Brilliant Blue), CI77491 (also titanium dioxide, E-171), CI77499 and CI77492 (also iron oxides, E-172).

In such a case, it is preferred according to the invention if the lipstick preparation comprises the colorants in a total concentration of 0.05% to 0.5% by weight, based on the total weight of the preparation.

If the preparation comprises CI15850 (also Red 6, E180 or Lithol Rubine) as sole colorant, then this compound is advantageously used according to the invention in a concentration of 0.05% to 0.4% by weight, based on the total weight of the preparation. If the preparation comprises a combination of CI15850 and CI42090, then the use concentration that is preferred according to the invention for CI15850 is from 0.05% to 0.4% by weight, based on the total weight of the preparation, and for CI42090 is from 0.02% to 0.15% by weight, based on the total weight of the preparation.

If the preparation comprises CI77491, then this compound is advantageously used according to the invention in a concentration of 0.02% to 0.1% by weight, based on the total weight of the preparation.

If the preparation comprises CI77499, then this compound is advantageously used according to the invention in a concentration of 0.02% to 0.1% by weight, based on the total weight of the preparation.

If the preparation comprises CI77492, then this compound is advantageously used according to the invention in a concentration of 0.005% to 0.05% by weight, based on the total weight of the preparation.

It is advantageous in the context of the present invention if the lipstick preparation comprises castor oil (INCI: Ricinus Communis Seed Oil).

According to the invention, for the process according to the invention, the pigments, inter alia, are preferably predispersed in this oil before they are added to the preparation.

It is preferred according to the invention if the preparation comprises castor oil in a concentration of 18% to 25% by weight, based on the total weight of the preparation.

Embodiments of the present invention that are advantageous according to the invention are also characterized in that the lipstick preparation comprises shea butter (INCI: Butyrospermum Parkii Butter), sunflower oil (INCI: Helianthus Annuus Hybrid Oil) and/or beeswax (INCI: Cera Alba), sunflower wax (Helianthus Annuus Seed Cera).

If the preparation comprises shea butter (INCI: Butyrospermum Parkii Butter), then this compound is advantageously used according to the invention in a concentration of 2% to 5% by weight, based on the total weight of the preparation.

If the preparation comprises sunflower oil (INCI: Helianthus Annuus Hybrid Oil), then this compound is advantageously used according to the invention in a concentration of 45% to 55% by weight, based on the total weight of the preparation.

If the preparation comprises sunflower wax (Helianthus Annuus Seed Cera), then this compound is advantageously used according to the invention in a concentration of 7% to 11% by weight, based on the total weight of the preparation.

Moreover, lip care products that are advantageous according to the invention are characterized in that the preparation comprises cetyl palmitate, hydrogenated rapeseed oil (INCI: Hydrogenated Rapeseed Oil) and/or cetearyl alcohol. The use of hydrogenated rapeseed oil is preferred according to the invention here.

For cetyl palmitate, the use concentration that is advantageous according to the invention is from 2% to 10% by weight, based on the total weight of the preparation.

For cetearyl alcohol, the use concentration that is advantageous according to the invention is from 5% to 15% by weight, based on the total weight of the preparation.

For hydrogenated rapeseed oil (INCI: Hydrogenated Rapeseed Oil), the use concentration that is advantageous according to the invention is from 5% to 9% by weight, based on the total weight of the preparation.

Moreover, it is may be advantageous according to the invention if the lip care preparation comprises butylated hydroxytoluene (BHT). The use concentration according to the invention for this substance is from 0.05% to 0.15% by weight, based on the total weight of the preparation.

Furthermore, it is advantageous according to the invention if the lipstick preparation comprises tocopherol and/or ascorbyl palmitate. In particular, the use of tocopherol is preferred here. The use concentration according to the invention for this substance is from 0.05% to 0.15% by weight, based on the total weight of the preparation.

The lipstick formulation according to the invention may, advantageously according to the invention, comprise one or more UV filters.

In such a case, it is preferred according to the invention if the lipstick preparation comprises 4-(tert-butyl)-4′-methoxydibenzoylmethane (INCI: Butyl Methoxydibenzoylmethane) and/or 2,4,6-tris [anilino-(p-carbo-2′-ethyl-1′-hexyloxy)]-1,3,5-triazine (INCI: Ethylhexyl Triazone).

Last but not least, it is advantageous according to the invention if the proportion of hydrophilic constituents in the lipstick preparation is less than 5% by weight, based on the total weight of the preparation. Hydrophilic constituents are understood here to mean water and the water-soluble compounds.

According to the invention, to disperse these constituents, polyglyceryl esters such as Polyglyceryl-3 Diisostearate or Polyglyceryl-4 Diisostearate/Polyhydroxystearate/Sebacate are advantageously added to the preparation according to the invention. The use of Polyglyceryl-4 Diisostearate/Polyhydroxystearate/Sebacate is preferred according to the invention here.

The use concentration that is preferred according to the invention for Polyglyceryl-4 Diisostearate/Polyhydroxystearate/Sebacate here is 1.5% to 2.5% by weight, based on the total weight of the preparation.

Last but not least, the invention provides for the use of citric acid (INCI: Citric Acid) in cosmetic lipsticks or processes, such as those described in this disclosure, to increase the color stability and/or the emulsion stability of the preparation.

### COMPARATIVE EXPERIMENTS

The following formulations were produced and their appearance was determined optically. All use concentrations relate to % by weight, based on the total weight of the preparation.

All LAB values given below were measured with produced lip care sticks using the HunterLab ColorFlex EZ colorimeter. The sticks are slightly rotated out of the mechanism and placed upside down on the measuring opening.

| INCI | Example 1 | Example 2 | Example 3 |
|---|---|---|---|
| Sodium Saccharin | 1% | 1% | 1% |
| Neohesperidin | | | 0.5% |
| Dihydrochalcone | | | |
| Citric Acid | | 1% | |
| Glycerin | | | 1% |
| Aqua | 1% | 2% | 1.5% |
| Polyglyceryl-4 | 2% | 2% | 2% |
| Diisostearate/ | | | |
| Polyhydroxystearate/ | | | |
| Sebacate | | | |
| Helianthus Annuus | 9% | 9% | 9% |
| Seed Cera | | | |
| Hydrogenated | 7% | 7% | 7% |
| Rapeseed Oil | | | |
| Sunflower Hybrid Oil | 55.9% | 52.9% | 52.9% |
| Ricinus Communis | 20% | 20% | 20% |
| Seed Oil | | | |
| Butyrospermum | 4% | 4% | 4% |
| Parkii Butter | | | |
| Tocopherol | 0.1% | 0.1% | 0.1% |
| | Bulk was | Bulk was | Bulk was heated at 80° C. |
| | heated at 80° C. | heated at 80° C. | without agitation for 24 h. The |
| | without | without | polar phase separates from |
| | agitation for | agitation for | the oil phase and has a red |
| | 24 h. The | 24 h. The polar | discoloration. |
| | sodium | phase remains | Compound can be |
| | saccharin | dissolved and | homogenized again by |
| | crystallizes | separates from | mechanical action (shaking). |
| | and settles. | the oil phase. | Cast lipsticks exhibited the |
| | | Compound can | following LAB values: |
| | | be | L: 64.22 |
| | | homogenized | a: 4.27 |
| | | again by | b: 6.73 |
| | | mechanical | Here, the a value shows a |
| | | action | noticeable red tint in |
| | | (shaking). | comparison with the samples |
| | | | of Example 5-8. |

| INCI | Example 4 | Example 5 | Example 6 |
|---|---|---|---|
| Sodium | 1% | 1% | 1% |
| Saccharin | | | |
| Neohesperidin | 0.5% | 0.5% | 0.5% |
| Dihydrochalcone | | | |
| Citric Acid | 0.01% | 0.05% | 0.10% |
| Glycerin | 1% | 1% | 1% |
| Aqua | 1.51% | 1.55% | 1.6% |
| Polyglyceryl-4 | 2% | 2% | 2% |
| Diisostearate/ | | | |
| Polyhydroxystearate/ | | | |
| Sebacate | | | |
| Helianthus | 9% | 9% | 9% |
| Annuus Seed | | | |
| Cera | | | |
| Hydrogenated | 7% | 7% | 7% |
| Rapeseed Oil | | | |
| Sunflower | 52.88% | 52.8% | 52.7% |
| Hybrid Oil | | | |
| Ricinus | 20% | 20% | 20% |
| Communis Seed | | | |
| Oil | | | |
| Butyrospermum | 4% | 4% | 4% |
| Parkii Butter | | | |
| Tocopherol | 0.1% | 0.1% | 0.1% |
| | Bulk was heated at | Bulk was heated at | Bulk was heated at |
| | 80° C. without | 80° C. without | 80° C. without agitation |
| | agitation for 24 h. | agitation for 24 h. | for 24 h. The polar |
| | The polar phase | The polar phase | phase seperates from |
| | separates from the | separates from the | the oil phase. |
| | oil phase and has a | oil phase. | Compound can be |
| | red-orange | Compound can be | homogenized again by |
| | discoloration. | homogenized again | mechanical action |
| | Compound can be | by mechanical | (shaking). |
| | homogenized again | action (shaking). | Cast lipsticks exhibited |
| | by mechanical action | Cast lipsticks | the following LAB |
| | (shaking). | exhibited the | values: |
| | Cast lipsticks | following LAB | L: 70.23 |
| | exhibited the | values: | a: −0.60 |
| | following LAB | L: 68.72 | b: 6.96 |
| | values: | a: −0.09 | The a value is |
| | L: 66.19 | b: 7.43 | measurably lower than |
| | a: 3.00 | The a value is | for the samples with red |
| | b: 7.52 | measurably lower | discoloration (Example |
| | Here too there is a | than for the | 3-4). |
| | high a value in | samples with red | |
| | comparison with | discoloration | |
| | Example 5-8, which | (Example 3-4). | |
| | indicates a red | | |
| | discoloration. | | |

| INCI | Example 7 | Example 8 | Example 9 |
|---|---|---|---|
| Sodium Saccharin | 1% | 1% | 5% |
| Neohesperidin | 0.5% | 0.5% | 2.5% |
| Dihydrochalcone | | | |
| Citric Acid | 0.25% | 1% | 0.01% |
| Glycerin | 1% | 1% | 5% |
| Aqua | 1.75% | 2% | 7.51% |
| Polyglyceryl-4 | 2% | 2% | 2% |
| Diisostearate/ | | | |
| Polyhydroxystearate/ | | | |
| Sebacate | | | |
| Helianthus Annuus | 9% | 9% | 9% |
| Seed Cera | | | |
| Hydrogenated | 7% | 7% | 7% |
| Rapeseed Oil | | | |
| Sunflower Hybrid Oil | 52.4% | 51.4% | 36.88 |
| Ricinus Communis | 20% | 20% | 20% |
| Seed Oil | | | |
| Butyrospermum | 4% | 4% | 4% |
| Parkii Butter | | | |
| Tocopherol | 0.1% | 0.1% | 0.1% |
| | Bulk was heated at | Bulk was heated at | Bulk was heated at |
| | 80° C. without | 80° C. without | 80° C. without |
| | agitation for 24 h. | agitation for 24 h. | agitation for 24 h. |
| | The polar phase | The polar phase | The polar phase |
| | separates from the | separates from the | separates from the |
| | oil phase. | oil phase. Compound | oil phase and has a |
| | Compound can be | can be homogenized | red-orange |
| | homogenized again | again by mechanical | discoloration. |
| | by mechanical | action (shaking). | Compound can be |
| | action (shaking). | Cast lipsticks | homogenized |
| | Cast lipsticks | exhibited the | again by |
| | exhibited the | following LAB | mechanical action |
| | following LAB | values: | (shaking). |
| | values: | L: 71.78 | Cast lipsticks |
| | L: 69.28 | a: −0.46 | exhibited the |
| | a: −0.73 | b: 7.23 | following LAB |
| | b: 7.28 | The a value is | values: |
| | The a value is | measurably lower | L: 66.92 |
| | measurably lower | than for the samples | a: 1.24 |
| | than for the | with red discoloration | b: 8.95 |
| | samples with red | (Example 3-4). | Here, the a value |
| | discoloration | | shows a noticeable |
| | (Example 3-4). | | red tint in |
| | | | comparison with |
| | | | Example 10-11. |

| INCI | Example 10 | Example 11 | Example 12 |
|---|---|---|---|
| Sodium Saccharin | 5% | 5% | 10% |
| Neohesperidin | 2.5% | 2.5% | 5% |
| Dihydrochalcone | | | |
| Citric Acid | 0.25% | 0.5% | |
| Glycerin | 5% | 5% | 10% |
| Aqua | 7.75% | 8% | 15% |
| Polyglyceryl-4 | 2% | 2% | 2% |
| Diisostearate/ | | | |
| Polyhydroxystearate/ | | | |
| Sebacate | | | |
| Helianthus Annuus | 9% | 9% | 9% |
| Seed Cera | | | |
| Hydrogenated | 7% | 7% | 7% |
| Rapeseed Oil | | | |
| Sunflower Hybrid Oil | 36.4% | 35.9% | 16.9% |
| Ricinus Communis | 20% | 20% | 20% |
| Seed Oil | | | |
| Butyrospermum | 4% | 4% | 4% |
| Parkii Butter | | | |
| Tocopherol | 0.1% | 0.1% | 0.1% |
| | Bulk was heated at | Bulk was heated at | Bulk was heated |
| | 80° C. without | 80° C. without | at 80° C. without |
| | agitation for 24 h. | agitation for 24 h. | agitation for 24 h. |
| | The polar phase | The polar phase | The polar phase |
| | separates from the | separates from the | separates from |
| | oil phase. | oil phase. Compound | the oil phase and |
| | Compound can be | can be homogenized | has a red |
| | homogenized again | again by mechanical | discoloration. |
| | by mechanical action | action (shaking). | Compound can |
| | (shaking). | Cast lipsticks | be homogenized |
| | Cast lipsticks | exhibited the | again by |
| | exhibited the | following LAB | mechanical |
| | following LAB | values: | action (shaking). |
| | values: | L: 68.90 | Cast |
| | L: 69.89 | a: −0.64 | exhibited the |
| | a: −0.49 | b: 8.59 | following LAB |
| | b: 9.53 | The a value is | values: |
| | The a value is | measurably lower | L: 64.08 |
| | measurably lower | than for the samples | a: 1.51 |
| | than for the samples | with red discoloration | b: 11.99 |
| | with red discoloration | (Example 9). | Here, the a value |
| | (Example 9). | | shows a |
| | | | noticeable red tint |
| | | | in comparison |
| | | | with Example 13- |
| | | | 15. |

| INCI | Example 13 | Example 14 | Example 15 |
|---|---|---|---|
| Sodium Saccharin | 10% | 10% | 10% |
| Neohesperidin | 5% | 5% | 5% |
| Dihydrochalcone | | | |
| Citric Acid | 0.25% | 0.5% | 1% |
| Glycerin | 10% | 10% | 10% |
| Aqua | 15.25% | 15.5% | 16% |
| Polyglyceryl-4 | 2% | 2% | 2% |
| Diisostearate/ | | | |
| Polyhydroxystearate/ | | | |
| Sebacate | | | |
| Helianthus Annuus | 9% | 9% | 9% |
| Seed Cera | | | |
| Hydrogenated | 7% | 7% | 7% |
| Rapeseed Oil | | | |
| Sunflower Hybrid Oil | 16.4% | 15.9% | 14.9% |
| Ricinus Communis | 20% | 20% | 20% |
| Seed Oil | | | |
| Butyrospermum Parkii | 4% | 4% | 4% |
| Butter | | | |
| Tocopherol | 0.1% | 0.1% | 0.1% |
| | Bulk was heated at | Bulk was heated at | Bulk was heated |
| | 80° C. without | 80° C. without | at 80° C. without |
| | agitation for 24 h. | agitation for 24 h. | agitation for 24 h. |
| | The polar phase | The polar phase | The polar phase |
| | separates from the | separates from the | separates from |
| | oil phase. | oil phase. | the oil phase. |
| | Compound can be | Compound can be | Compound can |
| | homogenized again | homogenized again | be homogenized |
| | by mechanical | by mechanical | again by |
| | action (shaking). | action (shaking). | mechanical |
| | Cast lipsticks | Cast lipsticks | action (shaking). |
| | exhibited the | exhibited the | Cast lipsticks |
| | following LAB | following LAB | exhibited the |
| | values: | values: | following LAB |
| | L: 68.91 | L: 64.85 | values: |
| | a: −1.47 | a: −1.15 | L: 61.65 |
| | b: 10.32 | b: 9.99 | a: −0.80 |
| | The a value is | The a value is | b: 10.82 |
| | measurably lower | measurably lower | The a value is |
| | than for the | than for the | measurably lower |
| | samples with red | samples with red | than for the |
| | discoloration | discoloration | samples with red |
| | (Example 12). | (Example 12). | discoloration |
| | | | (Example 12). |

| INCI | Example 16 | Example 17 | Example 18 |
|---|---|---|---|
| Sodium Saccharin | 1% | 1% | 1% |
| Neohesperidin | 0.5% | 0.5% | 0.5% |
| Dihydrochalcone | | | |
| CI15850 | 0.08% | 0.08% | 0.08% |
| CI42090 | 0.035% | 0.035% | 0.035% |
| Citric Acid | | 0.01% | 0.05% |
| Glycerin | 1% | 1% | 1% |
| Aqua | 1.5% | 1.51% | 1.55% |
| Polyglyceryl-4 | 2% | 2% | 2% |
| Diisostearate/ | | | |
| Polyhydroxystearate/ | | | |
| Sebacate | | | |
| Helianthus Annuus | 9% | 9% | 9% |
| Seed Cera | | | |
| Hydrogenated | 7% | 7% | 7% |
| Rapeseed Oil | | | |
| Sunflower Hybrid Oil | 53.785% | 53.765 | 53.685 |
| Ricinus Communis | 20% | 20% | 20% |
| Seed Oil | | | |
| Butyrospermum | 4% | 4% | 4% |
| Parkii Butter | | | |
| Tocopherol | 0.1% | 0.1% | 0.1% |
| | Bulk was heated at | Bulk was heated at | Bulk was heated at |
| | 80° C. without | 80° C. without | 80° C. without |
| | agitation for 24 h. | agitation for 24 h. | agitation for 24 h. |
| | The polar phase | The polar phase | The polar phase |
| | separates from the | separates from the | separates from the |
| | oil phase together | oil phase. Oil phase | oil phase. |
| | with the pigments. | appears slightly | Compound can be |
| | Oil phase appears | greenish. | homogenized |
| | greenish before | Compound can be | again by |
| | shaking. | homogenized again | mechanical action |
| | Compound can be | by mechanical | (shaking). |
| | homogenized again | action (shaking). | Cast lipsticks |
| | by mechanical action | Cast lipsticks | exhibited the |
| | (shaking). | exhibited the | following LAB |
| | Cast lipsticks | following LAB | values: |
| | exhibited the | values: | L: 33.04 |
| | following LAB | L: 33.46 | a: 14.86 |
| | values: | a: 16.09 | b: 4.19 |
| | L: 34.86 | b: 3.12 | |
| | a: 18.21 | | |
| | b: 3.95 | | |

| INCI | Example 19 | Example 20 | Example 21 |
|---|---|---|---|
| Sodium Saccharin | 1% | 1% | 1% |
| Neohesperidin Dihydrochalcone | 0.5% | 0.5% | 0.5% |
| CI15850 | 0.08% | 0.08% | 0.08% |
| CI42090 | 0.035% | 0.035% | 0.035% |
| Citric Acid | 0.10% | 0.25% | 1% |
| Glycerin | 1% | 1% | 1% |
| Aqua | 1.6 | 1.75% | 2.5% |
| Polyglyceryl-4 Diisostearate/ | 2% | 2% | 2% |
| Polyhydroxystearate/Sebacate | | | |
| Helianthus Annuus Seed Cera | 9% | 9% | 9% |
| Hydrogenated Rapeseed Oil | 7% | 7% | 7% |
| Sunflower Hybrid Oil | 53.585% | 53.285% | 52.285% |
| Ricinus Communis Seed Oil | 20% | 20% | 20% |
| Butyrospermum Parkii Butter | 4% | 4% | 4% |
| Tocopherol | 0.1% | 0.1% | 0.1% |
| | Bulk was heated | Bulk was heated | Bulk was |
| | at 80° C. without | at 80° C. without | heated at |
| | agitation for 24 h. | agitation for | 80° C. without |
| | The polar phase | 24 h. The polar | agitation for |
| | separates from | phase separates | 24 h. The |
| | the oil phase | from the oil | polar phase |
| | together with the | phase. | separates |
| | pigments. | Compound can | from the oil |
| | Compound can be | be homogenized | phase. Bulk |
| | homogenized | again by | appears |
| | again by | mechanical | slightly |
| | mechanical action | action (shaking). | bluish. |
| | (shaking). | Cast lipsticks | Compound |
| | Cast lipsticks | exhibited the | can be |
| | exhibited the | following LAB | homogenized |
| | following LAB | values: | again by |
| | values: | L: 30.07 | mechanical |
| | L: 32.12 | a: 13.03 | action |
| | a: 13.87 | b: 0.49 | (shaking). |
| | b: 2.01 | | Cast lipsticks |
| | | | exhibited the |
| | | | following |
| | | | LAB values: |
| | | | L: 31.19 |
| | | | a: 11.62 |
| | | | b: −0.88 |

The Lab values of Examples 16-21 show that with increasing concentration of citric acid, the a and b value decrease steadily and thus cause a color shift into the blue-green range. The color thus obtained is optically preferred in comparison with the original color without citric acid.

| INCI | Example 22 | Example 23 | Example 23 |
|---|---|---|---|
| Sodium Saccharin | 1% | 1% | 1% |
| Neohesperidin Dihydrochalcone | 0.5% | 0.5% | 0.5% |
| CI77491 | 0.025% | 0.025% | 0.025% |
| CI77499 | 0.025% | 0.025% | 0.025% |
| CI77492 | 0.00875% | 0.00875% | 0.00875% |
| Citric Acid | | 0.125% | 0.25% |
| Glycerin | 1% | 1% | 1% |
| Aqua | 1.5% | 1.625% | 1.75% |
| Polyglyceryl-4 Diisostearate/ | 2% | 2% | 2% |
| Polyhydroxystearate/ | | | |
| Sebacate | | | |
| Helianthus Annuus Seed Cera | 9% | 9% | 9% |
| Hydrogenated Rapeseed Oil | 7% | 7% | 7% |
| Sunflower Hybrid Oil | 51.96625 | 51.71625% | 51.46625% |
| Ricinus Communis Seed Oil | 20.875% | 20.875% | 20.875% |
| Butyrospermum Parkii Butter | 4% | 4% | 4% |
| Tocopherol | 0.1% | 0.1% | 0.1% |
| Theobroma Cacao Seed Butter | 1% | 1% | 1% |
| | Bulk was heated | Bulk was heated | Bulk was |
| | at 80° C. without | at 80° C. without | heated at |
| | agitation for 24 h. | agitation for | 80° C. without |
| | Cast lipsticks | 24 h. | agitation for |
| | exhibited the | Cast lipsticks | 24 h. |
| | following LAB | exhibitedthe | Cast lipsticks |
| | values: | following LAB | exhibited the |
| | L: 49.71 | values: | following |
| | a: 12.34 | L: 47.84 | LAB values: |
| | b: 9.44 | a: 18.54 | L: 49.25 |
| | | b: 16.08 | a: 17.25 |
| | | | b: 15.30 |

Examples 22-23 show that with increasing concentration of citric acid, the a and b value increase steadily and thus cause a color shift into red-yellow. Here too, the color obtained after addition of citric acid is optically preferred.

### Examples

The examples that follow are intended to illustrate the present invention without limiting it. Unless stated otherwise, all amounts, proportions and percentages are based on the weight and the total amount or on the total weight of the preparations.

| | | |
|---|---|---|
| Sodium Saccharin | 1% | 1% |
| Neohesperidin Dihydrochalcone | 0.5% | 0.5% |
| CI15850 | 0.08% | |
| CI42090 | 0.035% | |
| CI77491 | | 0.025% |
| CI77499 | | 0.025% |
| CI77492 | | 0.00875% |
| Citric Acid | 0.5% | 0.1% |
| Glycerin | 1% | 1% |
| Aqua | 2% | 1.625% |
| Polyglyceryl-4 Diisostearate/ | 2% | 2% |
| Polyhydroxystearate/ | | |
| Sebacate | | |
| Helianthus Annuus Seed Cera | 9% | 9% |
| Hydrogenated Rapeseed Oil | 7% | 7% |
| Sunflower Hybrid Oil | Compensation | Compensation |
| | to 100% | to 100% |
| Ricinus Communis Seed Oil | 20% | 20.875% |
| Butyrospermum Parkii Butter | 4% | 4% |
| Tocopheryl Acetate | 0.11% | |
| Tocopherol | 0.1% | 0.1% |
| Theobroma Cacao Seed Butter | | 1% |

1. A cosmetic lipstick comprising a combination of
a) sodium saccharin (INCI: Sodium Saccharin) and
b) citric acid (INCI: Citric Acid).

2. The cosmetic lipstick as claimed in claim 1, wherein the preparation comprises water and/or glycerin.

3. A process for producing a cosmetic lipstick, wherein
i) firstly sodium saccharin (INCI: Sodium Saccharin) is dissolved in water and
ii) in a separate vessel citric acid (INCI: Citric Acid) is dissolved in water,
iii) in a second step both aqueous solutions are successively combined with the fat phase and
iv) the combined solutions are mixed with the remaining ingredients of the preparation and homogenized.

4. The lipstick as claimed in claim 1, wherein the lipstick preparation comprises sodium saccharin in a concentration of 0.2% to 10% by weight, based on the total weight of the preparation.

5. The lipstick as claimed in claim 1, wherein the lipstick preparation comprises citric acid in a concentration of at least 0.05% by weight, based on the total weight of the preparation.

6. The lipstick as claimed in claim 1, wherein the lipstick preparation additionally comprises neohesperidin dihydrochalcone (INCI: Neohesperidin Dihydrochalcone).

7. The lipstick as claimed in claim 1, wherein the lipstick preparation additionally comprises neohesperidin dihydrochalcone (INCI: Neohesperidin Dihydrochalcone) in a concentration of 0.1% to 5% by weight, based on the total weight of the preparation.

8. The process as claimed in claim 3, wherein in step i) the neohesperidin dihydrochalcone is dissolved together with the sodium saccharin in the water/glycerin mixture.

9. The process as claimed in claim 8, wherein the water/glycerin mixture does not comprise more than 25% by weight of glycerin, based on the water/glycerin mixture.

10. The lipstick as claimed in claim 1, wherein the lipstick preparation comprises one or more colorants selected from the group of CI15850 (also Red 6, E180 or Lithol Rubine), CI42090 (also Blue 1, E133 or Brilliant Blue), CI77491 (also titanium dioxide, E-171), CI77499 and CI77492 (also iron oxides, E-172).

11. The lipstick as claimed in claim 10, wherein the lipstick preparation comprises the colorants in a total concentration of 0.05% to 0.5% by weight, based on the total weight of the preparation.

12. The lipstick as claimed in claim 1, wherein the lipstick preparation comprises castor oil (INCI: Ricinus Communis Seed Oil).

13. The lipstick as claimed in claim 1, wherein the lipstick preparation comprises shea butter (INCI: Butyrospermum Parkii Butter), sunflower oil (INCI: Helianthus Annuus Hybrid Oil) and/or beeswax (INCI: Cera Alba), sunflower wax (Helianthus Annuus Seed Cera).

14. The lipstick as claimed in claim 1, wherein the lipstick preparation comprises tocopherol and/or ascorbyl palmitate.

15. The lipstick as claimed in claim 1, wherein the lipstick preparation comprises 4-(tert-butyl)-4′-methoxydibenzoylmethane (INCI: Butyl Methoxydibenzoylmethane) and/or 2,4,6-tris [anilino-(p-carbo-2′-ethyl-1′-hexyloxy)]-1,3,5-triazine (INCI: Ethylhexyl Triazone).

16. The lipstick as claimed in claim 1, wherein the lipstick preparation comprises hydrophilic constituents in an amount of less than 5% by weight, based on the total weight of the preparation.

17. The lipstick as claimed in claim 1, wherein the lipstick preparation comprises Polyglyceryl-3 Diisostearate or Polyglyceryl-4 Diisostearate/Polyhydroxystearate/Sebacate.

18. The use of citric acid (INCI: Citric Acid) in cosmetic lipsticks as claimed in claim 1 to increase the color stability and/or the emulsion stability of the preparation.
